(19) United States
(12) Patent Application Publication
(10) Pub. No.: US 2007/0106116 A1
(43) Pub. Date: May 10, 2007

(54) ENDOSCOPE-SHAPE MONITORING SYSTEM

(75) Inventor: Hideo SUGIMOTO, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/557,536

(22) Filed: Nov. 8, 2006

(30) Foreign Application Priority Data
Nov. 9, 2005 (JP) P2005-324533

(51) Int. Cl. A61B 1/00; A61B 5/05

(52) U.S. Cl. 600/117; 600/118; 600/424

(57) ABSTRACT

An endoscope shape monitoring system that is used to grasp a shape of a flexible insertion portion is provided. The endoscope shape monitoring system includes a plurality of coils. The coils are used as a magnetic sensor. The coils are disposed on a flexible portion of the insertion portion. The plurality of coils are arranged at positions where the coils are not subjected to a bending stress that is induced when the flexible portion is bent.

### BACKGROUND OF THE INVENTION

1. Field of the Invention

The present invention relates to a system or to an apparatus that is used for monitoring the shape of an insertion portion or a flexible tube of an endoscope that is inserted inside a cavity or a hollow of an inspection object.

2. Description of the Related Art

It is beneficial for an endoscopic operator to grasp the shape of a flexible tube of an endoscope that is inserted inside a body. In particular, the visualization of the endoscope shape inside the body has a significant advantage when operating a lower intestinal endoscope, such as a colonoscope, since insertion of the flexible tube into a tortuous intestine is difficult. As a result, various types of endoscope-shape monitoring systems have been proposed.

A system that uses an alternating magnetic field for detecting the shape of a flexible tube of an endoscope is conventionally known. In this system, a plurality of coils are disposed along the longitudinal direction of the flexible tube, and a three-dimensional position and a direction for each of the coils are detected by using electromagnetic interactions between the alternating magnetic field and the coils. For example, the shape of the flexible tube is represented by a three-dimensional spline curve, which is obtained from positional data of measurement points where the coils are placed, and the result is displayed on a monitor.

### SUMMARY OF THE INVENTION

However, the coils that are provided inside the insertion portion have significant size so that the coils are repeatedly subjected to bending stress when the insertion portion is bent. Further, signal wires are wired between the coils and the operating portion of the endoscope, so that the signal wires are also subjected to bending stress and tensile stress. Therefore, the conventional endoscope-shape monitoring system has issues of durability.

Therefore, an object of the present invention is to improve the durability of an endoscope-shape monitoring system.

According to the present invention, an endoscope shape monitoring system that is used to grasp the shape of a flexible insertion portion is provided. The endoscope shape monitoring system includes a plurality of coils. The coils are used as a magnetic sensor. The coils are disposed on a flexible portion of the insertion portion. The plurality of coils are arranged at positions where the coils are not subjected to a bending stress that is induced when the flexible portion is bent.

According to another aspect of the present invention, an endoscope used in an endoscope shape monitoring system is provided. The endoscope shape monitoring system is used to grasp the shape of a flexible insertion portion that includes a bendable portion and a flexible portion. The endoscope includes a plurality of coils, and a plurality of rigid sections.

The coils are used as a magnetic sensor, and are disposed on the flexible portion of the insertion portion. The rigid sections have rigidity against the bending of the flexible portion. The rigid sections are arranged along the axis of the flexible portion at predetermined intervals, and the coils are disposed inside the rigid sections, so that the coils are protected from bending stress.

According to another aspect of the present invention, an endoscope used in an endoscope shape monitoring system is provided. The endoscope shape monitoring system is used to grasp the shape of a flexible insertion portion that includes a bendable portion and a flexible portion. The endoscope comprises a plurality of coils and a spiral band member.

The coils are used as a magnetic sensor, and are disposed on the flexible portion of the insertion portion. The spiral band member configures the flexible portion. The coils are integrally provided on the spiral band member, whereby the coils are not subjected to bending stress induced by bending of the flexible portion.

### BRIEF DESCRIPTION OF THE DRAWINGS

The objects and advantages of the present invention may be better understood from the following description, with reference to the accompanying drawings in which:

FIG. 1 is a general view of an endoscope to which an endoscope shape monitoring system as a first embodiment of the present invention is applied;

FIG. 2 schematically illustrates an arrangement of coils provided inside an insertion portion;

FIG. 3 is a partially magnified schematic perspective view, where one of the coils is detailed to illustrate the arrangement of the magnetic sensor coils;

FIG. 4 is a block diagram that shows overall electrical structures of the electronic endoscope system;

FIG. 5 schematically illustrates the structure of an insertion portion that is wound by a spiral band member;

FIG. 6 indicates a situation where the bendable portion is slightly bent;

FIG. 7 indicates a situation where the bendable portion is bent, where the end face of the distal end portion is turned around approximately 180 degrees;

FIG. 8 illustrates an example of an image representation of the shape of the insertion portion where the points P1-P8 are connected by segments (a linear interpolation);

FIG. 9 illustrates an example of an image representation of the shape of the insertion portion, where the points P1-P8 form the basis of a Bézier curve or a spline curve;

FIG. 10 schematically illustrates an example of structures of a bendable portion and a flexible portion;

FIG. 11 schematically illustrates another example of structures of the bendable portion and the flexible portion;

FIG. 12 schematically shows the shape of the bendable portion that is bent by a plurality of curvatures;

FIG. 13 indicates the positions of the points P1-P4 and the representation of the linear interpolation thereof, where the bendable portion 12B is bent in a narrow arc;

FIG. 14 schematically illustrates actual shapes of the bendable portion in several bending situations and relations of the positions between the point P1 and the point P2 in each of the bending situations; and

FIG. 15 schematically illustrates the relations between the positional coordinate data (X1, Y1, Z1) -(X9, Y9, Z9) and the bendable portion in situations where the point P1 is positioned at P1(0), P1(4), and P1(8),

### DESCRIPTION OF THE PREFERRED EMBODIMENTS

The present invention is described below with reference to the embodiments shown in the drawings.

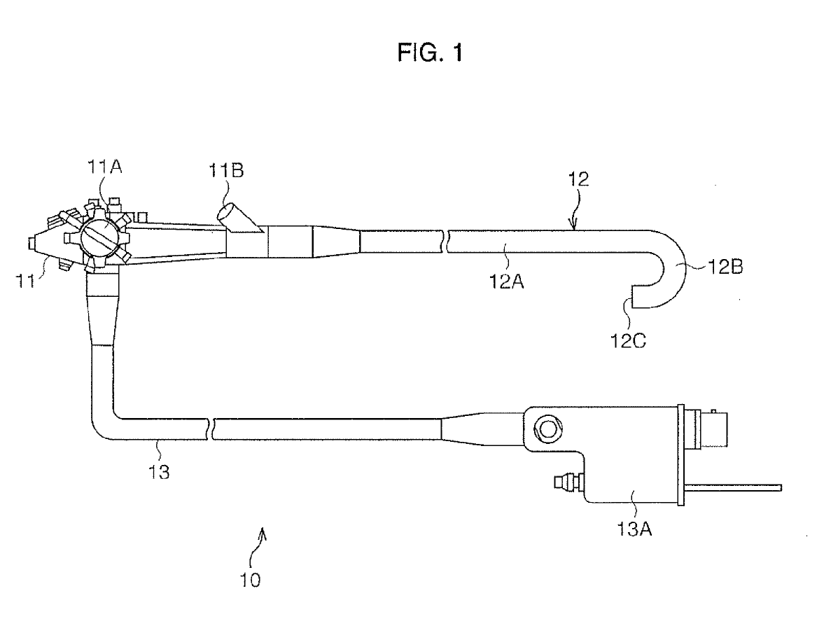

FIG. 1 is a general view of an endoscope to which a first embodiment of an endoscope-shape monitoring system of the present invention is applied. In this embodiment, an electronic endoscope is employed as an example of the endoscope.

The electronic endoscope 10 has an operating portion 11, which an endoscopic operator manipulates. An insertion portion (a flexible tube) 12 and a light-guide cable 13 are both connected to the operating portion 11. A connector 13A is provided at the distal end of the light-guide cable 13. The connector 13A is detachably attached to a processor apparatus (not depicted), for example, in which a light source and an image-signal processing unit are integrally installed. Namely, illumination light from the light source inside the processor apparatus is supplied to a cavity or a hollow viscus through the connector 13A of the electronic endoscope 10 and the light-guide cable 13. Further, image signals from the electronic endoscope 10 are supplied to the image-signal processing unit inside the processor apparatus.

The insertion portion 12 comprises of a flexible portion 12A, a bendable portion 12B, and a distal end portion 12C. Most of the insertion portion 12 is occupied by the flexible portion 12A that is formed of a flexible tube, which is freely bendable, and the flexible portion 12A is directly connected to the operating portion 11. The bendable portion 12B is provided between the distal end portion 12C and the flexible portion 12A, and is bended in accordance with a rotational operation of an angle lever 11A that is provided on the operating portion 11. For example, the bendable portion 12B can be bended as far as if the distal end portion 12C is rotated 180 degrees. Further, as is detailed later, the distal end portion 12C is provided with an imaging optical system, an imaging device, an illuminating optical system, and other components.

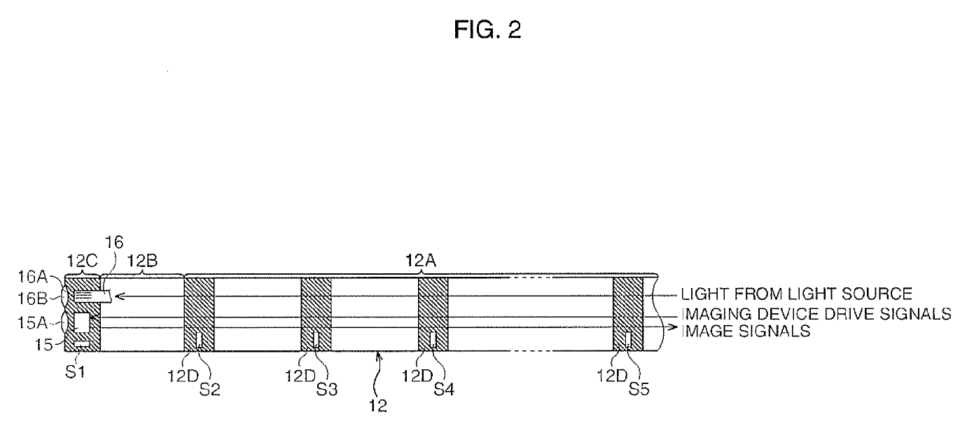

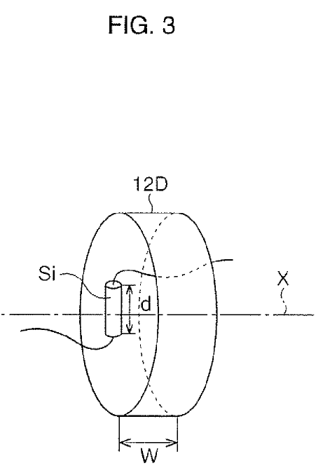

FIG. 2 schematically illustrates an arrangement of magnetic sensor coils installed inside the insertion portion 12. Further, FIG. 3 is a partially magnified schematic perspective view where one of the coils is detailed to illustrate the arrangement of the magnetic sensor coils. Note that, in FIG. 2, five magnetic sensor coils S1-S5 are shown as an example.

The distal end portion 12C of the insertion portion 12 is formed as a rigid section. Inside the distal end portion 12C, an imaging device 15 and the front end 16A of a light guide (optical fiber bundle) 16 are disposed. Further, an illuminating optical system 16B for emitting light from the light guide 16, and an imaging optical system 15A for projecting an object image onto the imaging device 15, are also provided in the distal end portion 12C of the insertion portion 12.

Although they are not shown in FIG. 2, a plurality of bending frame links that are linked together in a series are provided inside the bendable portion 12B. On the other hand, although the flexible portion 12A is wound with a spiral band member, a rigid section that shows rigidity against bending is not usually provided inside the flexible portion 12A. Therefore, the coils S2 to Sn (only S2-S5 are shown), which are provided inside the flexible portion 12A, are directly subjected to bending of the flexible portion 12A. In particular, the axis of the coils is conventionally arranged in parallel with the axis of the insertion portion, so that, in prior art, the coils tend to be subjected to a bending stress when the insertion portion is bended.

Therefore, in the first embodiment, rigid sections 12D that show rigidity against the bending are provided inside the flexible portion 12A at predetermined intervals. The coils S2-Sn are disposed, respectively, inside the rigid sections 12D and can be, for example, integrated with the rigid sections 12D. The number of rigid sections 12D may correspond to the number of the coils S2-Sn installed inside the flexible portion 12A.

For example, as shown in FIG. 3, the rigid sections 12D comprise hollow cylindrical members with a predetermined width W, and are formed of material, such as resin, having sufficient rigidity against banding of the flexible portion 12A. Further, material for the rigid sections 12D is selected from materials that do not affect the magnetic field around the magnetic sensor coils; in the present embodiment, hard plastic is used for the rigid sections 12D.

In the present embodiment, the coils S2-Sn are arranged inside the rigid sections 12D, so that the axis of the coil Si (where i=2, . . . , n) and the axis or the longitudinal direction of the insertion portion 12 are related by skew lines. In the present embodiment, the axis of the coil Si (where i=2, . . . , n) is disposed in a plane perpendicular to the central axis X of the insertion portion 12 (which comprises the cylindrical rigid sections 12D).

By arranging the coil Si as described above, the width W of the rigid sections 12D can be made narrower than the length “d” of the coil Si. Namely, obstruction or resistance against bending of the flexible portion 12A due to rigidity of the rigid sections 12D can be prevented by reducing the width W of the rigid sections 12D. In the present embodiment, the coil S1 disposed inside the distal end portion 12C is arranged in parallel with the central axis X, but this arrangement is only an example, and the arrangement is not restricted to this embodiment.

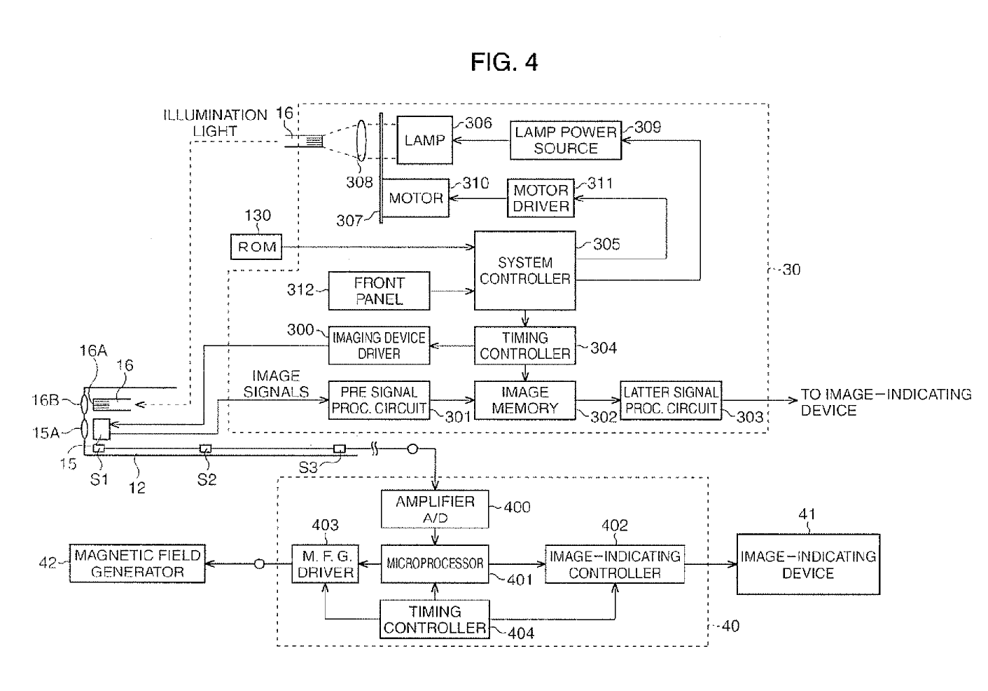

FIG. 4 is a block diagram that shows an electrical structure of the electronic endoscope system of the present embodiment. The electronic endoscope system of the present embodiment includes an insertion-portion-shape monitoring system that detects positions of the insertion portion 12 and indicates the shape thereof, and a capturing-image indicating system that captures an endoscopic image at the distal end of the insertion portion 12 and indicates the captured image.

The capturing-image indicating system generally includes the imaging device 15 and the light guide 16 that are provided inside the insertion portion 12, a processor unit 30, and an image-indicating device (not shown) for representing an image captured by the imaging device 15. The processor unit 30 supplies illumination light to the light guide 16, drives the imaging device 15, and processes the image signals from the imaging device 15.

On the other hand, the insertion-portion-shape monitoring system generally includes the plurality of coils S1-Sn, which are used as a magnetic sensor and are provided inside the insertion portion 12 of the endoscope, an insertion-portion-shape monitoring unit 40, an image-indicating device 41 for indicating the shape of the insertion portion 12, and a magnetic field generator 42.

In the present embodiment, the processor unit 30 and the insertion-portion-shape monitoring unit 40 are provided inside the processor apparatus to which the connector 13A (see FIG. 1) is detachably attached. Namely, the signal wires of the imaging device 15, the light guide cable 16, and the signal wires of the coils S1-Sn lead to the processor apparatus via the light guide cable 13 and the connector 13A.

The light guide 16 and the signal wires of the imaging device 15 are connected to the processor unit 30, provided inside the processor apparatus. The imaging device 15 is driven by an imaging device driver 300, provided inside the processor unit 30, and the image signals from the imaging device 15 are fed to a pre-signal processing circuit 301 of the processor unit 30.

The image signals that are subjected to predetermined image-signal processes at the pre-signal processing circuit 301 are temporarily stored in an image memory 302, and then successively fed to a latter signal processing circuit 303. At the latter signal processing circuit 303, the image signals are subjected to predetermined image-signal processes, and then the image signals are encoded as video signals. The video signals are fed to an output device, such as the image-indicating device.

Note that the imaging device driver 300 and the image memory 302 are driven by control signals from a timing controller 304, and a system controller 305 controls the timing controller 304.

Further, the imaging device 15 captures images inside the body, while emitting illumination light from the light guide 16. The illumination light is supplied from the light source unit inside the processor apparatus to the light guide 16. The light source unit includes a lamp 306, and white light from the lamp 306 is concentrated upon the end face of the light guide 16, which is inserted inside the processor apparatus, via a shutter 307 and a condenser lens 308.

The lamp 306 receives electric power from a lamp power source 309. A motor 310 that is controlled by a motor driver 311 drives the shutter 307. The lamp power source 309 and the motor driver 311 are controlled by the system controller 305.

Note that the system controller 305 is connected to a front panel 312, which includes switches that are operated by a user. The system controller 305 is able to change various types of preset parameters and modes according to operations of the switches on the front panel 312.

Further, a ROM 130 is provided inside the connector 13A of the electronic endoscope 10. When the connector 13A is attached to the processor apparatus, the ROM 130 is connected to the system controller 305, so that electronic endoscope identification information stored in the ROM 130 is transmitted to the system controller 305. Namely, the ROM 130 stores information relating to the electronic endoscope 10, such as the type of the scope and parameters used in the image processing and the information acquired by the system controller 305.

On the other hand, the signals from the coils (magnetic sensors) S1-Sn are amplified by a predetermined gain, and converted from analog signals to digital signals at an amplifier A/D 400. The signals of the coils S1-Sn, is which are converted to digital signals at the amplifier A/D 400, are input to a microprocessor 401, and the position of each coil S1-Sn is calculated.

Image data for representing the entire shape of the insertion portion 12 are generated at an image-indicating controller 402, based on the positions of the coils S1-Sn, which are calculated by the microprocessor 401, and output to the image-indicating device 41. The image data may represent the shape of the insertion portion 12 by using an interpolation curve line that connects the positions of the coils S1-Sn.

The positions of the coils S1-Sn are obtained by detecting the effects of electromagnetic interactions to the coils S1-Sn, where the effects are induced by the alternating magnetic field. For example, as is known in the art, the magnetic field generator 42 generates alternating magnetic fields in turn for each of the X, Y, and Z coordinates of an orthogonal coordinate system XYZ. The magnetic field generator 42 is controlled by a magnetic field generator driver 403. Further, the microprocessor 401, the image-indicating controller 402, and the magnetic field generator driver 403 are controlled by the timing controller 404.

As described above, according to the first embodiment of the present invention, the rigid sections are disposed in the flexible portion, where the magnetic sensor coils are disposed, and each coil is provided inside the rigid sections. Thereby, the coils are released from the stresses induced by the bending of the flexible portion; thus, the durability of the coils is improved.

Further, in the first embodiment, the width required for each rigid section is reduced by arranging the magnetic sensor coils in the plane perpendicular to the central axis of the insertion portion. Thereby, the rigid sections can be provided for the flexible portion without decreasing the flexibility.

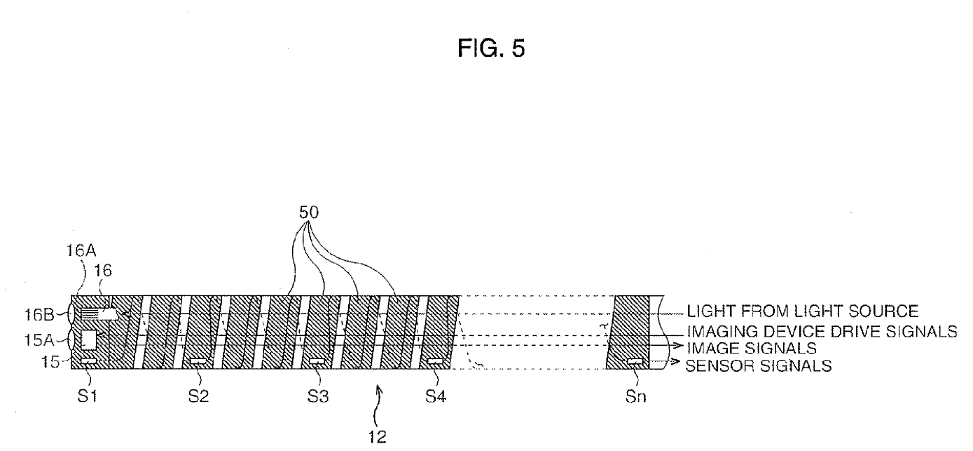

Next, with reference to FIG. 5, a second embodiment of the present invention is explained below. Although the structures of the second embodiment are dissimilar from those of the first embodiment in the aspect of mounting the magnetic sensor coils on the insertion portion, the remaining structures are the same as those in the first embodiment. The explanations will only be given for the dissimilar structures. Note that FIG. 5 schematically illustrates the structures of the insertion portion, which is wound with a spiral band member.

As described in the previous part of this specification, the insertion portion 12, including the flexible part, is wound with the spiral band member that forms a flexible tube. The spiral band member 50 is configured from a long band member, which is helically wound. The spiral band member 50 has a certain degree of rigidity in the lateral direction of the band, so that the insertion portion is bent or curved by a continuous subtle twist along the longitudinal direction of the band.

In the prior art, the magnetic sensor coils S2-Sn are disposed inside the flexible portion 12A, separate from the spiral band member 50, so that the coils S2-Sn may be arranged between neighboring band sections, or may come into contact with the other members provided inside the flexible tube or the spiral band member 50; thus, the coils S2-Sn can be affected by bending stress induced when the flexible portion 12A is bent. Further, the signal wires are connected to the coils S2-Sn, while the distances between each of the coils S2-Sn varies according to the manner of bending of the flexible portion 12A, so that the signal wires may be subjected to tensile stress when the insertion portion 12A is bent.

Therefore, in the second embodiment, as shown in FIG. 5, the magnetic sensor coils S1-Sn are integrally provided on the spiral band member 50, and the signal wires 51 are wired along the spiral band member 50. For example, coils S1-Sn and the signal wires 51 are previously attached to the spiral hand member 50 and integrated thereto. The flexible tube of the insertion portion 12 is configured by spirally winding the spiral band member 50, in which the coils S1-Sn are integrally provided. The coils S1-Sn may be arranged in the lateral direction of the spiral band member 50.

As described above, according to the second embodiment, the magnetic sensor coils and the signal wires are released from the stress induced by the flexible portion's bending; thus, the durability is improved as well as in the first embodiment.

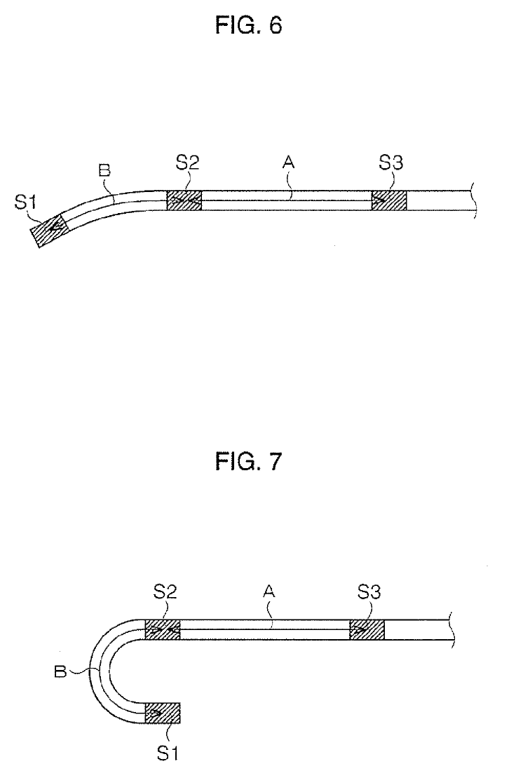

With reference to FIGS. 6-14, the processes for indicating the shape of the insertion portion are described below.

FIGS. 6 and 7 schematically illustrate the shapes of the endoscope insertion portion 12 around the distal end portion, when the angle lever 11A is operated and the bendable portion 12B is bent. FIG. 6 indicates a situation where the bendable portion 12B is slightly bent. FIG. 7 indicates a situation where the bendable portion 12B is bent in which the end face of the distal end portion 12C is turned around approximately 180 degrees.

In the present embodiment, the coil S1 (the first magnetic sensor) is provided in the distal end portion 12C of the insertion portion 12. The coil S2 (the second magnetic sensor) is disposed at an end of the bendable portion 12B, on the side close to the operational portion 11. Further, the coil S2 is separated from the coil S1 by a distance “B” along the axis. As it is described in the first and the second embodiments, the coils S3, . . . , Sn are successively arranged at the predetermined intervals A, from the side of the coils S2 to the side of the operational portion 11.

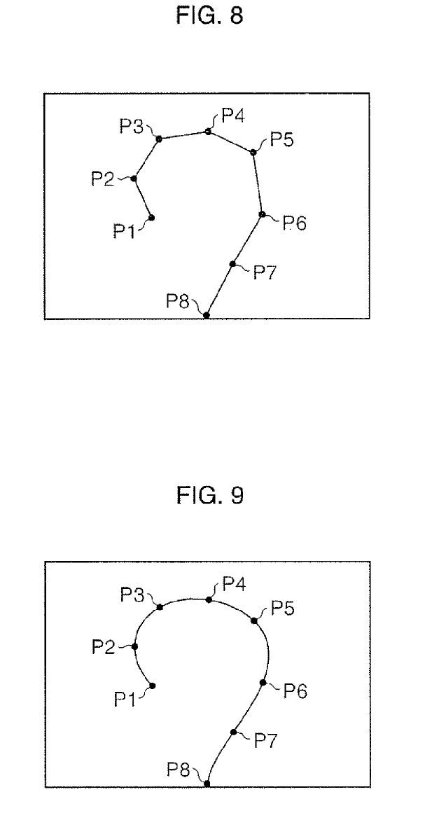

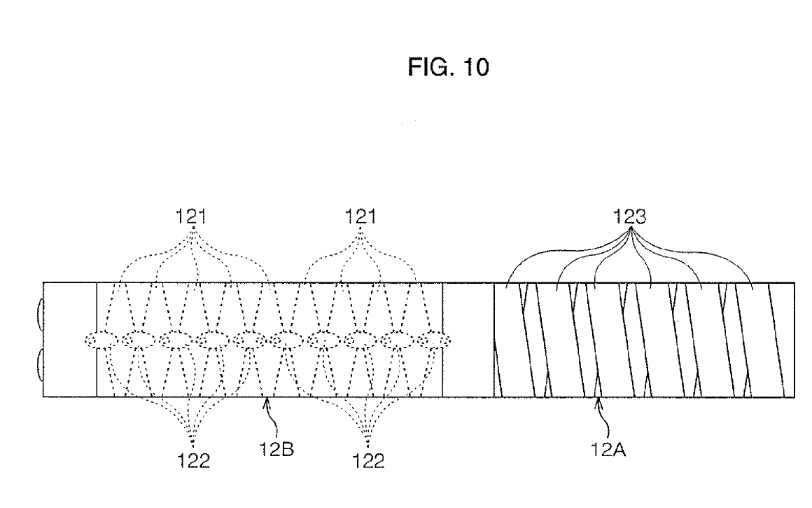

In the insertion-portion shape-indicating process, the shape of the insertion portion 12 is reproduced on the screen of the image-indicating device 41 by connecting the points P1-Pn that correspond to the positions of the coils S1-Sn, where the positions are obtained by using the alternative magnetic field. In FIG. 8, an example of image representation where the points P1-Pn are connected by segments (a linear interpolation) is illustrated. In FIG. 9, an example of image representation where the points P1-Pn are connected or fitted by a Bézier curve or a spline curve is illustrated.

However, the structures of the bendable portion 12B are generally different from those of the flexible portion 12A. Further, the way force acts on the bendable portion 12B is also different from the way that force acts on the flexible portion 12A, since the bendable portion 12B is affected by the force of the angle wires. Therefore, the manner of bending of the bendable portion 12B is quite different from that of the flexible portion 12A, so that if the same interpolation method were used for the flexible portion 12A and for the bendable portion 12B, in the conventional way, the reproduced shape of the bendable portion 12B could be quite different from the actual shape.

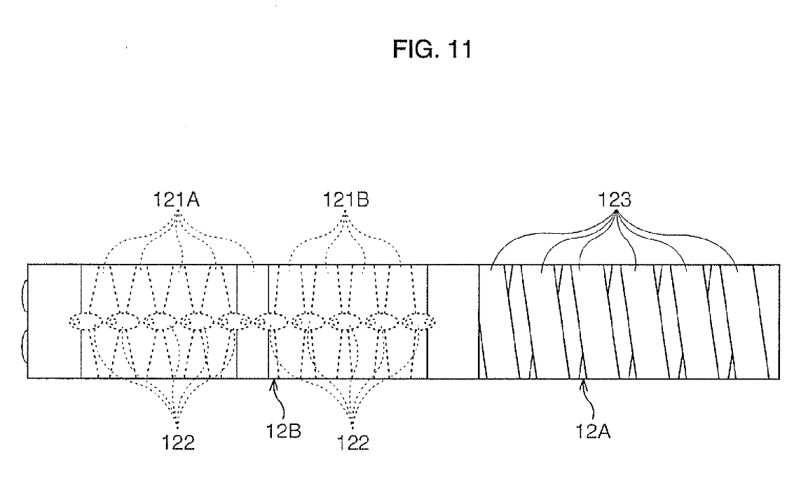

For example, as shown in FIG. 10, the flexible portion 12A is structured by a spiral band member 123, while the bendable portion 12B is structured by a plurality of bending frame links 121. Each of the neighboring bending frame links 121 is connected together with a hinge section 122, whereby to configure the bendable structure. Further, as an example, another structure of the bendable portion 12B is schematically shown in FIG. 11. In the example of FIG. 11, the bendable portion 12B includes two types of bending frame links 121A and 121B. In the example of FIG. 11, the bending frame links 121A, which have a narrower width than that of the bending frame links 121B, are arranged on the distal end side of the bendable portion 12B. Therefore, the distal end side of the bendable portion 12B can be bent by a relatively large curvature compared to the flexible portion side.

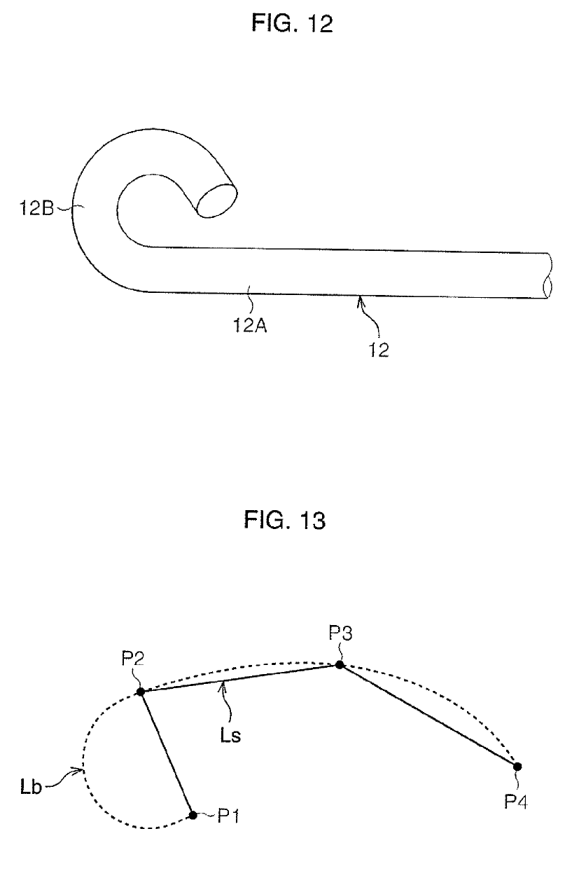

From the structures indicated in FIGS. 10 and 11, the curvature of the bendable portion 12B when the bendable portion 12B is factitiously bent by an operation of the angle lever 11A, is significantly larger than the curvature of the flexible portion 12A, which is due to a free bend. Further, the manner of bending of the bendable portion 12B is also quite dissimilar from that of the flexible portion 12A. For example, as shown in FIG. 12, when the bendable portion 12B is bent, the bendable portion 12B includes a plurality of curvatures, whose values are different from one another. Therefore, it is difficult to precisely represent the shape of the bendable portion 12B by applying the same method as used in the representation of the flexible portion 12A.

Referring to FIG. 13, the positions of the points P1-P4 and the representation of the linear interpolation thereof, when the bendable portion 12B is bent in a narrow arc, are indicated. Namely, the reproduced shape of the insertion portion 12, which is represented by the linear interpolation (where the points P1-P4 are connected by the segments), is described by the solid line Ls. On the other hand, the actual shape of the insertion portion 12 is described by the phantom line Lb.

As shown in FIG. 13, since the flexible portion 12A forms a gentle curve when it is bent, the reproduced shape (Ls) approximates the actual shape (Lb) for the intervals between the points P2-P4 that correspond to the flexible portion 12A. However, for the interval between the point P1 and the point P2 that corresponds to the bendable portion 12B, the reproduced shape is far from the actual shape. As an example of an extreme case, FIG. 13 represents the linear interpolation case. However, even by applying a Bézier curve or a spline curve for the interpolation, it would be difficult suitably to represent the shape of the bendable portion 12B when the bendable portion 12B is bent in a narrow arc, if the same interpolation method were used to represent the flexible portion 12A and the bendable portion 12B.

In order to reproduce the shape of the bendable portion 12B accurately, a plurality of magnetic sensor coils may be disposed inside the bendable portion 12B. However, a bending operation due to the manipulation of the angle lever 11A would be obstructed if a coil were disposed inside the bendable portion 12B, and the coil could also be damaged or destroyed. Accordingly, in the present embodiments, the coil S1 and the coil S2 are disposed on both ends of the bendable portion 12B.

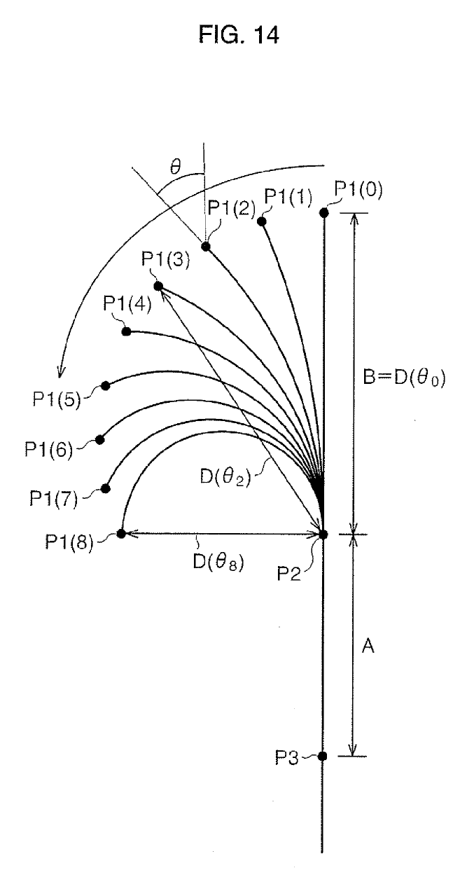

In general, the bending properties of the bendable portion 12B are specific for each product. The actual shapes of the bendable portion 12B in several bending situations, and relations of the positions between the point P1 and the point P2 in each of the bending situations are schematically illustrated in FIG. 14. In FIG. 14, nine types of bending situations of the bendable portion 12B are illustrated in stages from the non-bending situation to the situation where the bendable portion 12B is approximately turned around in the opposite direction.

The positions of the point P1 in each of the above nine bending situations are represented by P1(0)-P1(8). Further, the direction of the distal end portion 12C when the bendable portion 12B is being bent is represented by an angle “θ”, where the angle “θ” represents an angle against the direction of the distal end portion 12C, when the bendable portion 12B is directed straight forward and is not bent. Thus, the bending situation is represented by the angle “θ”. Namely, when the bendable portion 12B is not bent and the point P1 is positioned at P1(0), the angle θ=0°. Further, when the bendable portion 12B is bent such that the distal end portion 12C faces the opposite direction, and when the point P1 is positioned at P1(8), the angle θ=180°. Moreover, the angles “θ” for each of the positions P1(0)-P1(8) are represented by θ0-θ8.

In general, the distance “D” between the point P1 and the point P2 and the angle “θ” have a one-to-one correspondence (i.e., D=D(θ), θ=D⁻¹(D)). Further, when the distal end portion 12C is directed in a certain direction “θ”, the bendable portion 12B generally describes the same shape. Therefore, when the distance “D” is determined from the positions of the points P1 and P2, the shape of the bendable portion 12B can be determined.

In the present embodiments, information representing the correspondence between the distance “D” (the relative distance between the points P1 and P2) and the shape of the bendable portion 12B is stored in a memory, such as the ROM 130 (see FIG. 4), as bendable-portion shape data. Note that the shapes of the bendable portion 12B that correspond to the distances “D” are measured before hand. Examples of the bendable-portion shape data are shown in Table 1.

| | | | | | | | | | |
|---|---|---|---|---|---|---|---|---|---|
| P1 (0) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (1) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (2) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (3) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (4) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (5) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (6) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (7) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (8) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |

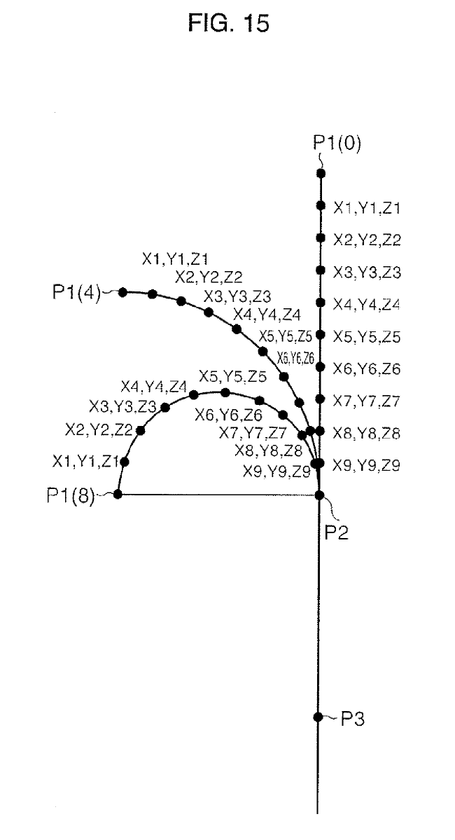

As shown in Table 1, the bendable-portion shape data, for example, include coordinates (x, y, z) of positions that are allocated along the central axis of the bendable portion 12B per a predetermined interval for each of the relative positions P1(0)-P1(8). As for the examples shown in Table 1, the positional coordinate data for the bendable portion 12B between the points P1 and P2 are given so as the interval between the points P1 and P2 is evenly divided into ten intervals. For each of the points P1(0)-P1(8), nine positional coordinate data (X1, Y1, Z1)-(X9, Y9, Z9) are stored. The correspondence between the positional coordinate data (X1, Y1, Z1)-(X9, Y9, Z9) and the bendable portion 12B is schematically illustrated in FIG. 15 for the situations where the point P1 is positioned at P1(0), P1(4), and P1(8).

As mentioned above, when the distance “D” is calculated, the position of the point P1 with respect to the point P2 is uniquely determined (the degree of freedom about the axis is not considered). Thereby, one of the positions P1(0)-P1(8) is selected in accordance with the determination, and the shape of the bendable portion 12B is reproduced based on the positional coordinate data (X1, Y1, Z1)-(X9, Y9, Z9) corresponding to the selected position.

The bendable-portion shape data in the present embodiments can be positional information relating to any predetermined positions between the points P1 and P2, and the information may also include a curvature of the bendable portion 12B for each situation. Further, an interpolation function or parameters thereof may also be used for reproducing the shape of the bendable portion 12B, so that the information of the interpolation function and the parameters may be stored in the memory for each of the distances “D”. Moreover, any combinations of the above methods may also be adopted.

Namely, in the insertion-portion shape-indicating process of the present embodiments, different interpolation methods are applied for each of the bendable portion 12B and the flexible portion 12A, so that the entire shape of the insertion portion 12 is represented by the combination thereof. Namely, as for the flexible portion 12A, each position of the coils is connected together with a Bézier curve or a spline curve, in the same way as conventionally. On the other hand, as for the bendable portion 12B and the distal end portion 12C, the shape is represented by the interpolation based on the given insertion-portion shape data and the relative positional relationship between the coils S1 and S2, which are provided on both ends of the bendable portion 12B, such as on the flexible portion 12A side and on the distal end portion 12C side.

Note that, when the Bézier curve or the spline curve is used for representing the flexible portion 12A, a control point for the point P2 of the interpolation curve of the flexible portion 12A is determined from the geometrical parameters, such as for the tangential line and the curvature, selected for the bendable portion 12B.

As described above, according to the present embodiment, in addition to the effects mentioned in the first and second embodiments, the shape of the bendable portion can be more accurately represented by a simple structure; thereby, the entire shape of the insertion portion can be reproduced accurately.

Further, in the present embodiments, the situations of the bendable portion is assumed to be uniquely determined by the distance between the coils S1 and S2, so that only the above distance is used to determine the condition or shape of the bendable portion, and the corresponding bendable-portion shape data are referenced. However, the directions of the coils may also be used for determining the situation of the bendable portion, if differences among the above distances are not sufficient to determine the situation.

In the present embodiment, the alternating magnetic field is generated outside the endoscope, by the magnetic field generator disposed outside an inspection object, and the coils and the magnetic sensors are disposed inside the insertion portion. However, the coils for generating a magnetic field can be disposed inside the insertion portion, and magnetic sensors can be disposed outside the insertion portion.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2005-324533 (filed on Nov. 9, 2005), which is expressly incorporated herein, by reference, in its entirety.

1. An endoscope shape monitoring system that is used to grasp a shape of a flexible insertion portion, the system comprising;
a plurality of coils that are used as a magnetic sensor, and that are disposed on a flexible portion of said insertion portion;
wherein said plurality of coils are arranged at positions where said coils are not subjected to bending stress induced by bending of said flexible portion.

2. The system as claimed in claim 1, wherein a plurality of rigid sections having rigidity against the bending of said flexible portion are arranged along the axis of said flexible portion at predetermined intervals, and said plurality of coils are disposed inside said plurality of rigid sections, so that said plurality of coils are protected from the bending stress.

3. The system as claimed in claim 2, wherein said rigid sections comprise a cylindrical member.

4. The system as claimed in claim 3, wherein the width of said rigid sections in the axial direction is greater than the length of said coils.

5. The system as claimed in claim 3, wherein the axis of said coils and the axis of said flexible portion are related by skew lines.

6. The system as claimed in claim 1, wherein said plurality of coils are integrally provided on a spiral band member that configures said flexible portion.

7. The system as claimed in claim 6, wherein signal wires of said coils are wired along said spiral band member.

8. The system as claimed in claim 1, wherein said system detects the positions of said plurality of coils by using an alternating magnetic field generated outside said endoscope.

9. An endoscope used in an endoscope shape monitoring system that is used to grasp a shape of a flexible insertion portion, said insertion portion including a bendable portion and a flexible portion, the endoscope comprising:
a plurality of coils that are used as a magnetic sensor, and that are disposed on said flexible portion of said insertion portion; and
a plurality of rigid sections having rigidity against the bending of said flexible portion;
wherein said plurality of rigid sections are arranged along the axis of said flexible portion at predetermined intervals, and said plurality of coils are disposed inside said plurality of rigid sections, so that said plurality of coils are protected from bending stress.

10. An endoscope used in endoscope shape monitoring system that is used to grasp a shape of a flexible insertion portion, said insertion portion including a bendable portion and a flexible portion, the endoscope comprising:
a plurality of coils that are used as a magnetic sensor, and that are disposed on said flexible portion of said insertion portion; and
a spiral band member that configures said flexible portion;
wherein said plurality of coils are integrally provided on said spiral band member, whereby said coils are not subjected to bending stress induced by bending of said flexible portion.
